(19) United States
(12) Patent Application Publication
(10) Pub. No.: US 2024/0066813 A1
(43) Pub. Date: Feb. 29, 2024

(54) NEEDLE TIP SHIELDING DEVICE, PROCESS FOR MANUFACTURING A THERMOPLASTICELEMENT

(71) Applicant: Greiner Bio-One GmbH, Kremsmuenster (AT)

(72) Inventor: Per Knutsson, Helsingborg (SE)

(21) Appl. No.: 18/280,203

(22) PCT Filed: Sep. 3, 2022
(86) PCT No.: PCT/EP2022/055288

§ 371 Date: Sep. 1, 2023

(30) Foreign Application Priority Data
Mar. 2, 2021 (SE) 2150232-3

(51) Int. Cl. B29C 71/02; B29C 35/16; B29C 45/72

(52) U.S. Cl. CPC B29C 71/02; B29C 35/16; B29C 45/7207; B29C 2071/022; B29L 2031/753

(57) ABSTRACT

A method for manufacturing a thermoplastic unit is described. The method may include injecting a thermoplastic material into a mold cavity. The method may also include curing the thermoplastic material to form the thermoplastic unit. The method may further include annealing the thermoplastic unit, after curing, at an annealing temperature under a T_{g} of the thermoplastic material.

### CROSS-REFERENCE TO RELATED APPLICATIONS

This application claims priority to International Patent Application No. PCT/EP2022/055288, filed on Mar. 2, 2022, and Swedish Patent Application No. SE 2150232-3, filed on Mar. 2, 2021, the contents of both of which are hereby incorporated by reference in their entirety.

### TECHNICAL FIELD

This invention pertains in general to the field of injection molding thermoplastic units, and more specifically injection manufacturing of thermoplastic units intended to be stored in a tension state, which is to be released during use of said unit. More particularly the invention relates to an injection or infusion needle tip shielding device, said shielding device having at least one arm suitable for being urged by a needle stem into said tension state and covering said needle tip when in a resting state. Furthermore, the present invention pertains to a units/items/products obtainable by such a method.

### BACKGROUND

In the field of medicine, such as within the field of devices for infusion and injection, it is known to arrange needle tip shielding devices on the injection or infusion needle, said shielding device having the ability to snap in front of the needle tip upon withdrawal of the needle. These needle tip shielding devices have historically been manufactured in stainless steel. After the manufacturing and packing of the devices for infusion and injection, the devices are sterilized for hygienic reasons.

Such a needle tip shielding device is for example disclosed in EP1003588. However, needle tip shielding devices will, when being arranged in for example a catheter hub, scratch and tear the polymeric catheter hub lumen, resulting in a major risk of flushing plastic material into the blood stream of the patient. Additionally, the manufacturing of such shielding devices of stainless steel is cumbersome and costly, since several punching and bending stations have to be used.

In WO2011036574 a shielding device with arms of a plastic material has been envisioned. However, due to the sterilizing step after injection molding, and arrangement on the needle in tension state, the plastic arms will relax and loose their tension. Hence, they will risk not to adequately cover the needle tip of the needle in the relaxed state. Therefore, a rubber band surrounds the arms, to help the arms to cover the needle tip upon withdrawal of the needle.

Hence, there is a need of an improved thermoplastic unit, and manufacturing method thereof, allowing for maintained stored tension even after having been subjected to a heating temperature after injection molding, such as a sterilizing step or storing and transportation during extreme heat. Specifically, there is a need for a needle tip shielding device and a manufacturing method thereof, and injection and infusion assemblies comprising such a shielding device, allowing for such maintained tension storing under heating after injection molding, such as a sterilizing step or storing and transportation during extreme heat.

### SUMMARY

Accordingly, the present invention preferably seeks to mitigate, alleviate or eliminate one or more of the above-identified deficiencies in the art and disadvantages singly or in any combination and solves at least the above mentioned problems by providing a method for manufacturing a thermoplastic unit, said method comprising the steps of: injecting a thermoplastic material into a mold cavity; curing said thermoplastic material to form said thermoplastic unit; and annealing said thermoplastic unit after said curing at an annealing temperature under T_{g} of said thermoplastic material.

Further features and advantages of the invention and its embodiments are set forth in the appended claims.

### BRIEF DESCRIPTION OF THE DRAWINGS

These and other aspects, features and advantages of which the invention is capable of will be apparent and elucidated from the following description of embodiments of the present invention, reference being made to the accompanying drawings, in which

FIG. 1 is a cross sectional view, along a longitudinal axis, of a catheter assembly with a needle shield arranged therein in a loaded state, according to one embodiment of the present invention; and

FIG. 2 is a cross sectional view, along a longitudinal axis, of a catheter assembly with a needle shield arranged therein in a released state, according to one embodiment of the present invention.

### DETAILED DESCRIPTION

The following description focuses on an embodiment of the present invention applicable to a method for manufacturing a thermoplastic unit, and in particular to a method for manufacturing a thermoplastic unit, which is to spring loaded during pre-treatment, transportation and storing, such that the spring load can be activated during use. En more specifically, the following description focuses on the manufacturing of a needle shield and a vascular catheter system comprising such a needle shield. However, it will be appreciated that the invention is not limited to this application but may be applied to the manufacturing of many other thermoplastic units, such as needle shields for vascular injection systems, thermoplastic valves, locking sprints etc.

During injection molding of a thermoplastic unit, such as a needle shield for a vascular catheter system, the thermoplastic material is heated before injection to a temperature well above its melting temperature. Under normal circumstances, such as if the thermoplastic material is polycarbonate (PC), the thermoplastic material is heated to 300 to 340° C.

Other thermoplastic materials may also be used in the method according to the present invention. The thermoplastic material may for example be selected from the group comprising acrylonitrile butadiene styrene (ABS), acrylonitrile styrene acrylate (ASA), cellulose acetate (CA), polyoxymethylene (POM), poly(methyl methacrylate) (PMMA), polybutylene terephthalate (PBTP), liquid crystal polymer (LCP), polyamide (PA), polysulfone (PSU), styrene acrylonitrile (SAN), polyetherimide (PEI), polyethersulfone (PES), polyamideimide (PAI), polycarbonate (PC), polyphenylene oxide/styrene butadiene (PPO/SB), a styrenic block copolymer, a polyolefinic mixture, an elastomeric alloy, a thermoplastic polyurethane, a thermoplastic copolyester, a thermoplastic polyamide, or combinations of these. The temperature during the pre-heating before injection molding is then suitable selected in accordance with the melting temperature of the selected thermoplastic material, which is within the knowledge of the skilled person.

A thermoplastic material may be an amorphous or crystalline thermoplastic material. Regardless if the thermoplastic material is an amorphous or crystalline thermoplastic, the thermoplastic material will loose its solid state internal structure when being heated well above its melting temperature. After heating the thermoplastic material above its melting temperature, i.e. melting, the thermoplastic material is injected into a mold cavity, wherein the thermoplastic material is rapidly cooled to below melting its melting point, such as normally to 50 to 100° C. The injection force combined with the rapid cooling will freeze the polymeric chains (molecules) in a stretched orientation in comparison with its ideal state, which may be a fully entangled (amorphous thermoplastics) or more or less crystalline (crystalline thermoplastics) structure. In this orientation the formed, injection molded unit/object, will have an inherent tension, due to this stretching of the polymeric chains/molecules. If the object/unit then is arranged in an even more strained position, such as with an arm or lip in a flexed position, and subsequently being subjected to heating close to or over the T_{g} of the relevant thermoplastic material, the polymeric chains/molecules will start to rearrange to strive towards a more equilibrium arrangement. When this is happening, also the tension caused by the flexed arrangement of the object/unit will be lost. Thus, during heat treatment or exposing to heat after injection molding and arrangement in a flexed/strained arrangement, such object/unit will loose or at least have a severely lowered spring effect in comparison with the desired spring effect. This will jeopardize the entire function of the object/unit.

To overcome these drawbacks, an annealing step—after injection molding but before positioning the object/unit in flexed state or tension state—comprising a heating step to a temperature, an annealing temperature, close to but under the T_{g} of the relevant thermoplastic material.

The annealing temperature is preferably selected to be in the interval of T_{g}−50° C. to T_{g}−5° C., such as 10 to 40 C below said T_{g}, such as 25 to 35 C below said T_{g}, somewhat depending and being selected in relation to the thermoplastic material in question. This temperature is high enough to ensure reformation of the polymeric chains on a micro-scale, to allow for release of inherent tension created by the injection molding, while being low enough for not jeopardizing the material to change structurally on a macro-scale due to being too close to the T_{g} of the thermoplastic material in question.

The time period for the annealing step may preferably be selected to be between 30 to 180 minutes, such as 60 to 180 minutes. This time interval is adequately selected to be long enough to release enough of the inherent tension caused by the injection molding while simultaneously being short enough for not jeopardizing structural changes of the object/unit.

In one specific embodiment, when T_{g} is in the interval of 100 to 200° C., the annealing temperature (Tₐ) may be selected to be 5 to 50° C. below said T_{g}. In another embodiment, when said thermoplastic material has a T_{g} in the interval of 120 to 170° C., said Tₐ is selected to be 10 to 40° C. below said T_{g}. In yet another embodiment, when said thermoplastic material has a T g in the interval of 140 to 160° C., said Tₐ is selected to be 25 to 35° C. below said T_{g}.

Below, a table of different thermoplastic materials, their T_{g}, and suitable Tₐ intervals is disclosed in form of Table 1.

TABLE 1

| | T_{g} | Tₐ | |
|---|---|---|---|
| PolyCarbonate (PC) | 150° C. | 120 ± 10° | C. |
| Acrylonitrile Butadiene Styrene (ABS) | 110° C. | 80 ± 10° | C. |
| Poly (Methyl Methacrylate) (PMMA) | 105° C. | 75 ± 10° | C. |
| Acrylonitrile Styrene Acrylate (ASA) | 100° C. | 70 ± 10° | C. |
| Styrene AcryloNitrile (SAN) | 100° C. | 70 ± 10° | C. |
| PolySulfone (PSU) | 195° C. | 165 ± 10° | C. |
| Cellulose Acetate (CA) | 120° C. | 90 ± 10° | C. |
| PolyPhenyleneOxide/StyreneButadine | 180° C. | 150 ± 10° | C. |
| (PPO/SB) | | | |
| PolyEtherSulfone (PES) | 230° C. | 200 ± 10° | C. |
| PolyEtherImide (PEI) | 215° C. | 185 ± 10° | C. |
| PolyAmideImide (PAI) | 280° C. | 250 ± 10° | C. |

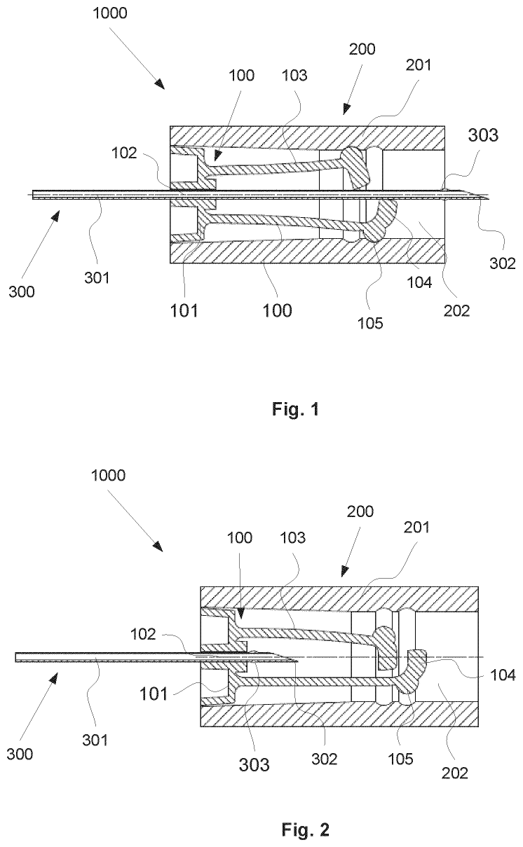

In a specific embodiment, the object/unit is a needle shield 100, in accordance with FIGS. 1 and 2. The needle shield 100 is intended to be arranged in a intravenous catheter assembly 1000 in a loaded state, in accordance with FIG. 1, to be released into a released state upon withdrawal of the needle, in accordance with FIG. 2. The catheter assembly 1000 includes the needle shield 100, a catheter unit 200 and a needle unit 300.

The catheter unit 200 comprises a catheter hub 201 and a catheter (not shown) extending distally from the catheter hub 201. The catheter is hollow and tubular, and configured to house a needle stem therein. The catheter is made of a suitable polymeric material. The catheter hub 201 is also made of a suitable polymeric material, such as polypropylene or polyethylene, which are cheap plastic materials with good injection molding properties. The hollow and tubular configuration of the catheter provides a lumen that is in flow communication with an interior cavity 202 of the catheter hub 201. The interior cavity 202 is positioned in the proximal end of the catheter hub 201, and the proximal opening into the interior cavity 202 may end in a luer fitting, such as a luer lock or luer slip, adapted to receive a tubing set, which in a known manner, administers intravenous fluid into the patient. The catheter unit 200 thus comprises a catheter hub 201 and a catheter extending distally from the catheter hub 201, said catheter having a lumen being in flow communication with an interior cavity 202 of the catheter hub 201.

The catheter is secured within an axial passageway in distal hub section by means of a sleeve received within passageway, which engages the proximal end of the catheter. This passageway communicates at its proximal end with interior cavity 202, which also acts as a flash chamber, formed in catheter hub 201. The distal end of the catheter may be tapered, to facilitate introduction into the vein of the patient.

The needle unit 300 of the catheter instrument 1000 comprises a needle hub. A needle 301 extends distally from the needle hub. The needle hub may have an axial opening for receiving the proximal end zone of the needle 301. The needle 301 comprises a needle shaft and a needle tip 302, said needle tip 302 forming the distal end point of the needle unit 300. The needle hub, as is conventional, may be hollow and may include a flash chamber at its proximal end. As is also conventional, the needle 301 is received within a hollow tubular catheter, the proximal end of which is concentrically affixed within the distal end of a catheter hub 201. At the distal end zone of the needle shaft, the needle 301 is provided with a bulge 304. The needle unit 300 thus comprises a needle hub and a needle 301 with a needle shaft and a needle tip 302 extending distally from the needle hub.

In the ready position of the catheter instrument 1000, the distal end of the needle hub is snugly received in the proximal end of the interior cavity 202 of the catheter hub 201, such that the needle 301 extends through the cavity 202, the passageway and distally beyond the catheter hub 201 and catheter so that the needle tip 302 extends beyond a the distal end of the catheter 202. Thus, the needle hub is connected to the proximal end of the catheter hub 201 and said needle shaft 301 is arranged in the lumen of the catheter, in a ready position of said catheter instrument 1000. The needle hub may be connected to the proximal end of the catheter hub 201 and said needle shaft 301 being arranged in the lumen of the catheter, in a ready position of said catheter instrument 1000.

In use, the distal tip 302 of the needle 301 and the catheter are inserted into a patient's vein. Thereafter, the health care practitioner manually places the catheter further into the vein and then withdraws the needle by grasping and moving by hand the proximal end of the needle unit 300. The luer of the catheter hub 201, in the proximal end of the cavity 202, is then fitted with a source of the fluid that is to be administered into the patient's vein.

The needle shield 100 is arranged inside the interior cavity 202 of the catheter hub 200. The needle shield 100 comprises a base plate 101. The base plate 101 is provided with a hole 102, extending there through, i.e. from the proximal side of the base plate 101 to the distal side of the base plate 101. Preferably, the hole 102 is arranged centrally on the base plate 101, such that arrangement of needle 301 through said hole 102 is facilitated while the needle 301 is arranged in accordance with the ready position of the catheter instrument 1000.

A first resilient arm 103 is extending distally from an attachment point at said base plate 101. Preferably, due to manufacturing reasons, the attachment point is located at the periphery of the base plate 101. The resilient arm 103 has a resting state, from which it may be urged to yield free passage for the needle 301 through said hole 102 in an axial direction of said base plate 101 in a tension state. This released resting state is disclosed in FIG. 2. The resilient arm 103 is in its tension state when the catheter instrument 1000 is in its ready position, in accordance with FIG. 1. The resilient arm 103 is adapted for clamping a needle tip 302 of a needle 301 extending through the hole 102 when the resilient arm 103 is in said resting state. For this reason, a straight imaginary line extending longitudinally through said hole 102 in the axial direction of said base plate 101 coincides with said at least one resilient arm 103 when said resilient arm 103 is in said resting state. This may be facilitated by providing the resilient arm 103 with a distal hook element 104, at the distal end of the resilient arm 103. The needle shield 100 may thus be arranged inside the interior cavity 202 of the catheter hub 201, and said needle being arranged through said hole 102 with the resilient arm 103 being urged into its tension state by said needle shaft. The resilient arm 103 is then dimensioned such that it may be flexed into its tension state when the catheter instrument 1000 is in its ready position.

The resilient arm 103 of said needle shield 100 may comprise a central portion 105 being urged by said needle shaft into retaining contact with an interior wall of said catheter hub 201. In this way the interaction between the catheter unit 200 and the needle shield device 100 may be broken once the needle 301 has been displaced proximally into a position where the distal end of the resilient arm 103 falls down in front of the needle tip 302, which in turn makes the central portion 105 to be displaced centrally. When the central portion 105 of the resilient arm 103 is displaced centrally, the needle shield 100 looses its contact with the interior wall of said catheter hub 201. Alternatively, the needle shield 100 is held in place in the catheter hub 201 through friction between the base plate 101 and the interior wall of the catheter hub 201.

After the distal tip 302 of the needle 301 and the catheter have been inserted into a patient's vein, the needle unit 300 is displaced proximally in relation to the catheter unit 200 and the needle shield 100. The needle shield 100 is retained in the catheter hub 201 of the catheter unit 200 through interaction between the base plate 101 or the central portion 105 of the resilient arm 103, in accordance with above. When the needle unit 300 is displaced proximally in relation to the catheter unit 200 and the needle shield 100, also the needle 301 is displaced proximally in relation to these two. Once the needle tip 302 passes proximally beyond the distal end of the resilient arm 103, such as the hook element 105, the distal end of the resilient arm 103 snaps in front of the needle tip 303. The bulge 303 on the needle shaft then hits the base plate 101, since the bulge 303 has been dimensioned with a somewhat larger diameter than the through hole 102 of the base plate 101. Also, the bulge 303 has been positioned on the needle shaft at a distance from the needle tip 30302 largely corresponding to the distance between the base plate 101 and the distal end of the resilient arm 103, such that the needle shield 100 may be secured at the distal end of the needle 301 once the needle tip 303 has been displaced proximally beyond the distal end, such as the hook element 105, of the needle shield 100. In this position, the needle shield 100 is released from the catheter unit 200 through overcoming the frictional force between the base plate 101 and the interior wall of the catheter hub 201 or by the central displacement of the central portion 105 of the resilient arm 103, in accordance with above.

The needle shield 100 may be provided with more than one resilient arm 103. An additional resilient arm may further stabilize the positioning of the needle tip shield 100 on the needle shaft 300. Also the second resilient arm may be provided with a distal hook element for central displacement once the needle tip 303 have passed proximally beyond the distal end of the first and second resilient arms 103.

The elements and components of an embodiment of the invention may be physically, functionally and logically implemented in any suitable way. Indeed, the functionality may be implemented in a single unit, in a plurality of units or as part of other functional units. As such, the invention may be implemented in a single unit, or may be physically and functionally distributed between different units and processors.

Although the present invention has been described above with reference to specific embodiments, it is not intended to be limited to the specific form set forth herein. Rather, the invention is limited only by the accompanying claims and, other embodiments than the specific above are equally possible within the scope of these appended claims.

In the claims, the term “comprises/comprising” does not exclude the presence of other elements or steps. Furthermore, although individually listed, a plurality of means, elements or method steps may be implemented by e.g. a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

1. A method for manufacturing a thermoplastic unit, the method comprising:
injecting a thermoplastic material into a mold cavity;
curing the thermoplastic material to form the thermoplastic unit; and
annealing the thermoplastic unit, after curing at an annealing temperature under a T_{g} of the thermoplastic material.

2. The method according to claim 1, wherein the thermoplastic material is an amorphous thermoplastic material.

3. The method according to claim 1, wherein:
the T_{g} of the thermoplastic material is from 100 to 200° C.; and
the annealing temperature is 5 to 50° C. below the T_{g}.

4. The method according to claim 3, wherein:
the T_{g} of the thermoplastic material is from 120 to 170° C.; and
the annealing temperature is 10 to 40° C. below the T_{g}.

5. The method according to claim 4, wherein:
the T_{g} of the thermoplastic material is from 140 to 160° C.; and
the annealing temperature is 25 to 35° C. below the T_{g}.

6. The method according to claim 1, wherein the thermoplastic unit is annealed for 30 to 180 minutes.

7. The method according to claim 6, wherein the thermoplastic unit is annealed for 60 to 180 minutes.

8. The method according to claim 1, wherein the thermoplastic material includes at least one of polyoxymethylene (POM), polybutylene terephthalate (PBTP), liquid crystal polymer (LCP), polyamide (PA), polysulfone (PSU), polyetherimide (PEI), polycarbonate (PC), polyphenylene oxide/styrene butadiene (PPO/SB), a styrenic block copolymer, a polyolefinic mixture, an elastomeric alloy, a thermoplastic polyurethane, a thermoplastic copolyester, and a thermoplastic polyamide.

9. The method according to claim 2, wherein:
the T_{g} of the thermoplastic material is from 100 to 200° C.; and
the annealing temperature is 5 to 50° C. below the T_{g}.

10. The method according to claim 9, wherein:
the T_{g} of the thermoplastic material is from 120 to 170° C.; and
the annealing temperature is 10 to 40° C. below the T_{g}.

11. The method according to claim 10, wherein:
the T_{g} of the thermoplastic material is from 140 to 160° C.; and
the annealing temperature is 25 to 35° C. below the T_{g}.

12. The method according to claim 1, further comprising placing the thermoplastic unit in at least one of a flexed state and a tensioned state.

13. The method according to claim 12, wherein the thermoplastic unit is annealed before placing the thermoplastic unit in the at least one of the flexed state and the tensioned state.

14. The method according to claim 1, wherein curing the thermoplastic material to form the thermoplastic unit includes rapidly cooling the thermoplastic material to a temperature below a melting point of the thermoplastic material.

15. The method according to claim 14, wherein rapidly cooling the thermoplastic material includes freezing polymeric chains of the thermoplastic material in a stretched orientation.

16. The method according to claim 1, wherein annealing the thermoplastic unit includes releasing inherent tension via reformation of polymeric chains of the thermoplastic material.

17. The method according to claim 1, wherein the thermoplastic unit is a needle shield for a vascular catheter system.

18. A method for manufacturing a thermoplastic unit, the method comprising:
injecting a thermoplastic material into a mold cavity;
curing the thermoplastic material to form the thermoplastic unit;
after curing the thermoplastic material, annealing the thermoplastic unit at an annealing temperature that is 5 to 50° C. below a glass transition temperature (T_{g}) of the thermoplastic material; and
after annealing the thermoplastic unit, placing the thermoplastic unit in at least one of a flexed state and a tensioned state.

19. The method according to claim 18, wherein the annealing temperature is 10 to 40° C. below the T_{g}.

20. The method according to claim 19, wherein the annealing temperature is 25 to 35° C. below the T_{g}.
